(19) United States
(12) Patent Application Publication
(10) Pub. No.: US 2007/0250062 A1
(43) Pub. Date: Oct. 25, 2007

(54) Intramedullary Nail

(76) Inventors: Javier Ara Pinilla, Santa Cruz de Tenerife (ES); Guillermo De La Barreda Lopez, San Cruz de Tenerife (ES); Donato Monopoli Forleo, San Cruz de Tenerife (ES)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/580,873

(22) PCT Filed: Nov. 25, 2003
(86) PCT No.: PCT/ES03/00597

§ 371 Date: Mar. 1, 2007

(51) Int. Cl. A61B 17/72

(52) U.S. Cl. 606/62

(57) ABSTRACT

The invention relates to an intramedullary nail. More specifically, the invention relates to a structure which combines a tubular nail (1-2-3-2′), a probe (4) which can move axially inside the tubular nail and a bone-fixing support (6), such as to form a head (1) on said nail, from which a plurality of thin rods (2) extend integrally. The rods are distributed over a considerably-long imaginary cylindrical surface having a reduced diameter and meet at a node A(3), beyond which they extend into segments (2′)j having an independent free end. According to the invention, a projecting part (5) of the probe (4) acts on the aforementioned segments (2′) when the probe is moved towards the head (1), in order to produce the radial deformation of the rods (2′) such as to enable same to penetrate the spongy bone tissue. When the protrusion (5) reaches the node (3) of the nail, said node (3) moves towards the head (1), thereby causing the radial expansion of the above-mentioned segment (2) of the rods. In this way, the rods adapt to the inner wall of the bone, exerting an elastic tension for improved fixing, said fixing necessitating only the screws that are used to stabilise the support (6) to which the head (1) of the nail is subsequently fixed internally.

### OBJECT OF THE INVENTION

The present invention relates to an intramedullary nail, of the type used to secure and immobilise fractures in long bones such as the femur.

The object of the invention is to achieve an elastic nail that is easier to implant in the bone, thus causing less damage thereto and improving the fixation, which also helps the bone to knit together.

### BACKGROUND OF THE INVENTION

Nails are usually used to immobilise a long fractured bone, being hammered into one of the ends of the bone and having at each end a pair of holes for receiving respective cross screws that immobilise the nail by securing it to the two parts of the bone and consequently joining said fractured parts to one other.

This solution has certain problems, particularly with regard to the following aspects:
As the bones to be immobilised are not straight, the nail tends to become deformed as it is hammered in, with the risk of passing through the bone, causing excessive damage to the spongy part thereof, which separates the intramedullary canal from the hard outer layer.
Its fixation necessitates the use four cross screws and it is quite difficult to line up said screws with the holes in the nail.
It is necessary to use x-rays or probe holes to locate the holes for the screws, as these are hidden inside the bone.

The difficulty of implanting said cross screws becomes considerably more pronounced in the case of the lower or distal holes, and the use of x-rays is not acceptable to surgeons, as they are frequently exposed to high radiations, particularly on their hands.

Although guiding systems exist in order to help to centre the holes, as do other systems with a “probe hole” wherein a guide is used to indicate the position of the holes in the nail, these solutions do not solve the above-described problems.

A nail called the “Marchetti” nail is known, which attempts to avoid such problems. It consists of a multiple nail, i.e. a plurality of very thin rods, which extend from a common core, so that they “open up” as they are inserted into the intramedullary canal and are driven into the spongy bone tissue, thus ensuring distal fixation without the need for distal nails.

More specifically, this set of thin rods is aided by a ring, which ascends when the nail is half inserted, opening the set of rods so that as they continue to be inserted into the intramedullary canal the thin rods point towards the spongy tissue and are driven into it.

Although this solution substantially simplifies the operating system and minimises the use of x-rays, it does present certain problems with regard to the following
It is not very secure.
The thin filaments or rods can damage the bone.
The fixation is arbitrary, as the deformation and manner in which the filaments are driven into the bone is not controllable.

### DESCRIPTION OF THE INVENTION

The intramedullary nail proposed by the invention successfully solves the above-described problems in each of the aspects that have been mentioned.

More specifically, to do this said nail consists of a functional combination of a nail and a probe that can move axially inside the nail, the purpose this probe being to cause a radial deformation of the nail, so that this need only be fixed by screws at the proximal end of the bone, whilst it is fixed at the distal end by said expansion effect.

This is achieved thanks to the fact that the nail itself has a plurality of filaments extending from a head at its proximal end, which are disposed according to an imaginary cylindrical surface and which converge at a node that is considerably distanced from the head, beyond which said filaments extend in a wide section, the probe including a marked protrusion close to its distal end, so that, once the nail-probe assembly has been implanted inside the bone, as the probe moves upwards said protrusion first causes a radial deformation of the ends of the filaments, which are driven into the spongy bone tissue, and when said protrusion reaches the node of the nail, it in turn causes said node to move towards the head of the nail, which in turn causes the initial section to bulge outwards, thereby adapting and fixing the filaments to the inner wall of the bone.

In order to achieve the aforementioned effect it is necessary for the upward movement of the probe to begin before the nail has fully penetrated the bone, so that after the radial expansion of the free end of the filaments, these are driven into the bone as the nail in turn completes its final forward movement.

In accordance with the description above, it is only necessary to perform one screwing operation, namely the operation of screwing the head of the nail to the proximal end of the bone, which can be done using a complementary template, without the need for x-rays.

### DESCRIPTION OF THE DRAWINGS

To complement this description and in order to aid a better understanding of the invention's characteristics, according to a preferred practical embodiment thereof, there is a set of illustrative and non-limiting drawings integral to said description, which are as follows:

FIG. 1. Shows a schematic perspective view of an intramedullary nail according to the object of the present invention.

FIG. 2. Shows a detail of a longitudinal section of the support for the head of the nail.

FIG. 3. Shows a detail of a perspective view of the nail without its support and without the interior probe.

FIG. 4. Shows a detail of a perspective view of the tool for fixing said support, in a working position at the corresponding end of the bone.

FIG. 5. Shows another perspective view of the nail assembly, here duly implanted in a femur.

### PREFERRED EMBODIMENT OF THE INVENTION

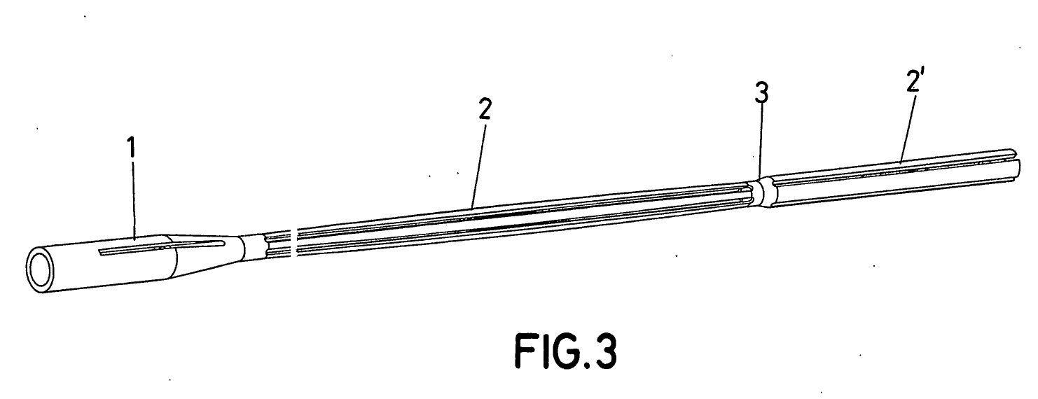

In view of the aforementioned figures, and particularly FIG. 3, it is possible to observe how the intramedullary nail proposed by the invention consists of a nail formed by a head (1) to which a plurality of rods (2) of a considerable length are joined and from which they extend, being distributed according to an imaginary cylinder of a small diameter and converging at a node (3), beyond which said rods (2) extend in terminal sections (2′) of considerable length with independent free ends.

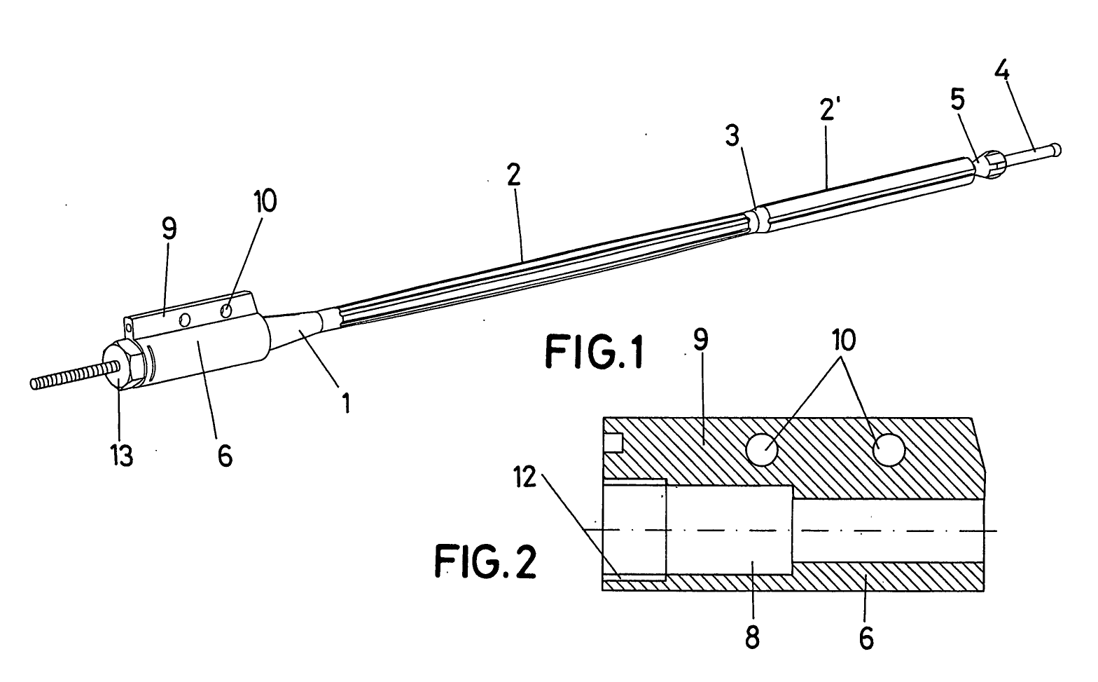

A probe (4) works with the nail (1-2), this probe consisting of a threaded rod that can be housed inside the hollow interior of the nail, having a protrusion (5) close to its distal end that acts as an expanding element for the terminal section (2′) of the rods (2), as will be seen below, the threaded rod (4) emerging through the proximal end of the nail, as can be observed in FIG. 1.

A support (6) is solidly fixed to the bone (7) by means of screws and the nail passes therethrough, finally and immovably fixing the head thereof, for which said support (6) has a stepped axial hole (8) to receive said head (1), also having a radial fin that has a pair of holes (10) through which the respective locking screws (11) pass.

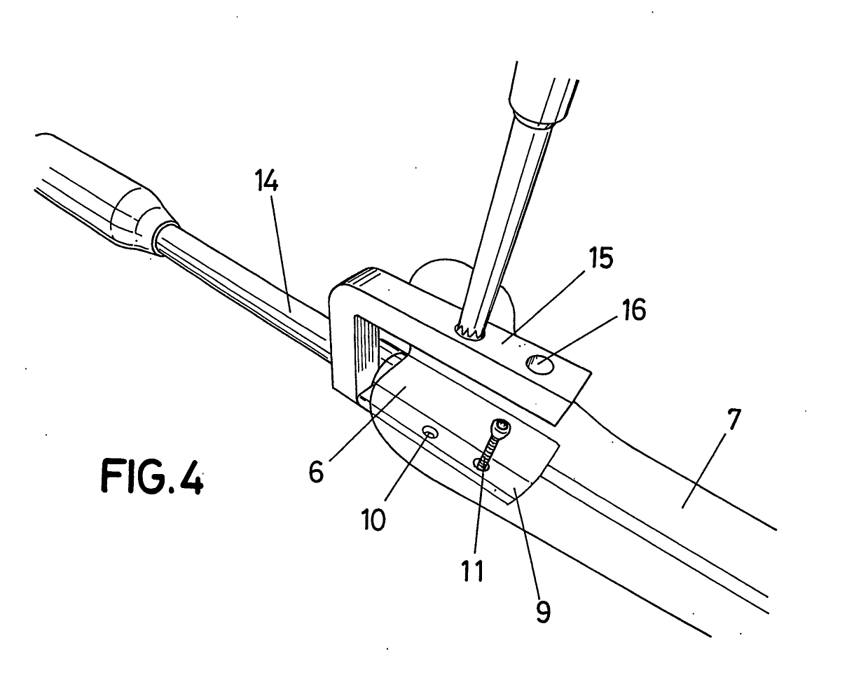

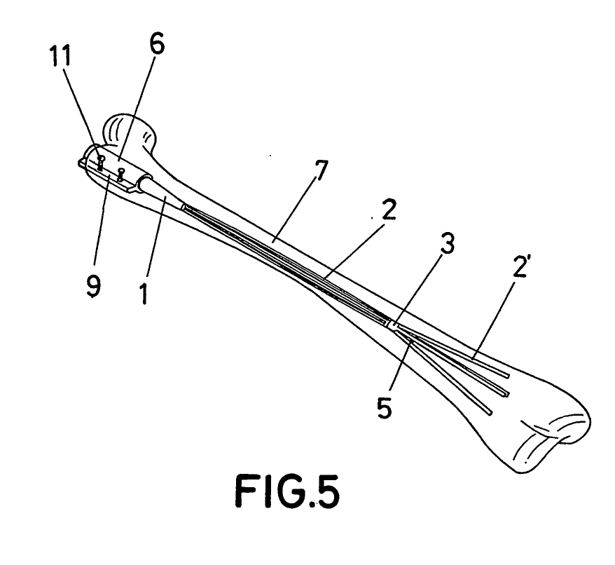

Said hole (8) in the support (6) includes a threaded section (12) at its outer end for the attachment of a collar (13) by means of which the axial traction on the probe (4) is finally performed, and which is initially used for the attachment of a tool (14), shown in FIG. 4, with a bent arm (15) and a pair of holes (16), so that when said tool (14) is duly attached to the support (6), the holes (16) in said tool are coaxially aligned with the holes (10) in the support (1), thus making it possible to drill holes in the bone (7) with the certainty that the screws (11) will therethrough inevitably reach the holes (10) in the support.

To assemble the nail, the support (6) is initially fixed to the proximal end of the fractured bone (7) and is screwed in place, then the assembly consisting of the probe (4) and nail (1-2) is inserted until it reaches a position in which a relative axial movement will occur between the probe (4) and the nail, giving rise to a first phase of divergence of the ends (2′) of the rods (2). At that moment, an actuation on the collar (13) causes an axial traction of the probe (4) until it reaches a position in which the protrusion (5) thereon comes into contact with the node (3) of the nail, thereby causing the terminal section (2′) of the rods to adopt their maximum divergence and press against the inner wall of the bone.

At this moment, the assembly of the head (1) on the support (6) is completed until it reaches a position in which there will be a longitudinal forward movement of the nail, so that the free ends of the rods (2′) are driven into the spongy bone tissue, and the proximal section (2) of said rods bulges outwards, i.e. said rods undergo a radial expansion in this area, pressing against the side wall of the bone and thus achieving not only an anti-rotational or anti-torsion tension, but also a longitudinal tension of the bone, which helps it to knit together.

The filaments that are drive into the spongy tissue are controllable and almost reach a perpendicular position relative the bone, which gives the nail greater stability.

The elastic properties of the nail generate longitudinal tension when the patient puts weight on the leg, thus helping the bone to knit together.

The use of x-rays is practically reduced to the follow-up stage, without the need for radiation while the screw is being implanted and, consequently, without said radiation affecting the surgeon's hands.

The special configuration of the nail and the manner in which it is assembled enable it to be implanted in the bone by pressure using a suitable nail driver, rather than the classic hammering methods.

1. Intramedullary nail, which is specially designed to secure and immobilise fractures in long bones such as the femur, characterised in that it consists of the functional combination of a tubular nail (1-2-3-2′) and a probe (4) that can move axially inside the nail, which includes a head (1) from which a plurality of thin rods (2) of a considerable length extend, which are grouped according to an imaginary cylindrical surface and converge towards the node (3), beyond which they extend in considerably wide sections (2′) that are independent at their free ends, whilst the probe (4) includes a protrusion (5) close to its distal end, which is initially situated outside the nail and first causes the radial deformation of the terminal section (2′) of the rods (2) during the axial movement of the probe relative to the nail and then causes the node (3) to move towards the head (1), which in turn causes a radial expansion of the nail in the proximal area of its rods (2).

2. Intramedullary nail, according to claim 1, characterised in that a support (6) works with the head (1) of the nail, being the only element of the assembly that is fixed by screws to the bone, specifically at the proximal end thereof, this support (6) having a stepped axial hole (8) for attachment of the head (1) and a radial fin (9) with a pair of holes (10) for screwing the support to the bone.

3. Intramedullary nail, according to claim 2, characterised in that inside the axial hole (8) in the support (6), specifically at the outer end thereof, there is a threaded section (12) for the attachment of a template for drilling into the bone, which is situated in line with the holes (10) of the support (6) and for the subsequent implantation of a collar (13) that can move the threaded rod (4) that constitutes the probe in order to displace the protrusion (5) thereon towards the head (1) of the nail.

4. Intramedullary nail, which is specially designed to secure and immobilise fractures in long bones such as the femur, characterised in that it consists of the functional combination of a tubular nail and a probe at least coextensive in length with the nail and movable axially inside the nail, said probe including a head, a plurality of thin rods of a considerable length extending from said head and having an intermediate node, said rods being grouped according to an imaginary cylindrical surface and converging towards the node independent at their free ends, said probe including a protrusion close to its distal end, which is adapted to initially extend beyond the nail and upon withdrawal to within the nail, causes the radial deformation of the terminal section of the rods during the axial withdrawal of the probe through the nail and then causes the node to move towards the head which in turn causes a radial expansion of the nail in the proximal area of the rods between the head and said node.
